(19) United States
(12) Patent Application Publication
(10) Pub. No.: US 2018/0368806 A1
(43) Pub. Date: Dec. 27, 2018

(54) ULTRASONIC DIAGNOSTIC APPARATUS

(71) Applicant: CANON MEDICAL SYSTEMS CORPORATION, Otawara-shi (JP)

(72) Inventors: Shingo TOYODA, Utsunomiya (JP); Hideo Onodera, Nasushiobara (JP); Shinichiro Kikuchi, Otawara (JP); Teruki Hagihara, Nasushiobara (JP); Tomohiro Fujita, Nasushiobara (JP)

(73) Assignee: CANON MEDICAL SYSTEMS CORPORATION, Otawara-shi (JP)

(21) Appl. No.: 16/014,487

(22) Filed: Jun. 21, 2018

(30) Foreign Application Priority Data
Jun. 23, 2017 (JP) 2017-123154

(51) Int. Cl. A61B 8/00

(52) U.S. Cl. CPC A61B 8/4218; A61B 8/14; A61B 8/44; A61B 8/40

(57) ABSTRACT

According to one embodiment, the ultrasonic diagnostic apparatus includes a main body housing, an arm, a device, a table, and an avoiding mechanism. The arm is mounted to the main body housing so as to be capable of ascending and descending and pivotable about a predetermined axis. The device is supported by the arm so as to be capable of ascending and descending and pivotable according to the movement of the arm. The table has a surface which is positioned in a descending direction of the arm and the device, and an object is placed on the surface. The avoiding mechanism changes a relative position between the object and a support position of ascending and descending movement of the arm such that interference between the object and at least one of the arm and the device due to the descending movement of the arm is avoided.

### CROSS-REFERENCE TO RELATED APPLICATIONS

This application claims the benefit of priority of Japanese Patent Application No. 2017-123154, filed Jun. 23, 2017, the entire contents of which are incorporated herein by reference.

### FIELD

Embodiments described herein relate generally to an ultrasonic diagnostic apparatus.

### BACKGROUND

Some ultrasonic diagnostic apparatuses have objects including peripheral devices such as printers, recording/reproducing apparatuses such as DVD recorders, placed near the main body of the apparatus. The ultrasonic diagnostic apparatus having a peripheral device placed near the main body, can be connected to the peripheral device by wire or wirelessly and can record or save the ultrasonic diagnostic image in the peripheral device.

Meanwhile, some ultrasonic diagnostic apparatus is configured to support a device such as a monitor and an operation panel via an arm, and the device supported by the arm is movable vertically and laterally on the front side of the main body of the apparatus via the arm. According to the ultrasonic diagnostic apparatus having this kind of arm, an user can easily change the position and posture of the monitor and the operation panel.

However, when the objects like peripheral devices are installed in the vicinity of the main body of the ultrasonic diagnostic apparatus having the arm, there are cases where the arm and the objects, or the monitor and the operation panel, collide with each other when the arm is moved. For example, when an object is placed on the right side or the left side of the main body of the apparatus so as to prevent a collision with the arm, depending on the positional relationship between the bed and the main body of the apparatus, the distance between the bed and the main body of the apparatus is increased by being disturbed by the object. When the object is installed on the rear side of the main body of the apparatus so as to prevent a collision with the arm, the distance from the object to the user operating the device is increased, and the convenience of the object including the peripheral device is greatly deteriorated.

In addition, when the object is installed in the descending direction of the arm, it is very inconvenient because it is difficult to lower the arm since the object limits the movement of the arm, whereby it suffers great restrictions on the range of change of the position and posture of the monitor and the operation panel.

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view showing an example of an ultrasonic diagnostic apparatus according to the present embodiment;

FIG. 2 is an explanatory diagram showing a configuration example of a rail as an example of an avoiding mechanism;

FIG. 3A is a plan view showing an example of a state in which interference between an arm or the like and a peripheral device due to a descending movement of the arm is avoided by the peripheral device moving along the rail;

FIG. 3B is a front view of FIG. 3A;

FIG. 4 is an explanatory diagram showing a configuration example of a movable arm support base as another example of the avoiding mechanism;

FIG. 5 is an explanatory diagram showing an example of a case where a rack-and-pinion mechanism is used as an interlocking mechanism;

FIG. 6 is an explanatory diagram showing an example of a case where a mechanism utilizing position information of the arm or the like is used as the interlocking mechanism;

FIG. 7 is an explanatory diagram showing an example of a case where a slide pin and a cam are used as a limiting movement mechanism;

FIG. 8 is an explanatory diagram showing an example of a relationship between the arm and the slide pin when the slide pin is within a range of the lower side of the inner periphery of the cam;

FIG. 9 is an explanatory diagram showing an example of a relationship between the arm and the slide pin when the slide pin is within a range of the right oblique side; and

FIG. 10 is a block diagram showing a configuration example of a lock mechanism.

### DETAILED DESCRIPTION

Hereinbelow, a description will be given of an ultrasonic diagnostic apparatus according to embodiments of the present invention with reference to the drawings.

In general, according to one embodiment, the ultrasonic diagnostic apparatus includes a main body housing, an arm, a device, a table, and an avoiding mechanism. The arm is mounted to the main body housing so as to be capable of ascending and descending and pivotable about a predetermined axis. The device is supported by the arm so as to be capable of ascending and descending and pivotable according to the movement of the arm. The table has a surface which is positioned in a descending direction of the arm and the device supported by the arm and an object is placed on the surface. The avoiding mechanism changes a relative position between the object and a support position of ascending and descending movement of the arm such that interference between the object and at least one of the arm and the device supported by the arm due to the descending movement of the arm is avoided.

### (Entire Configuration)

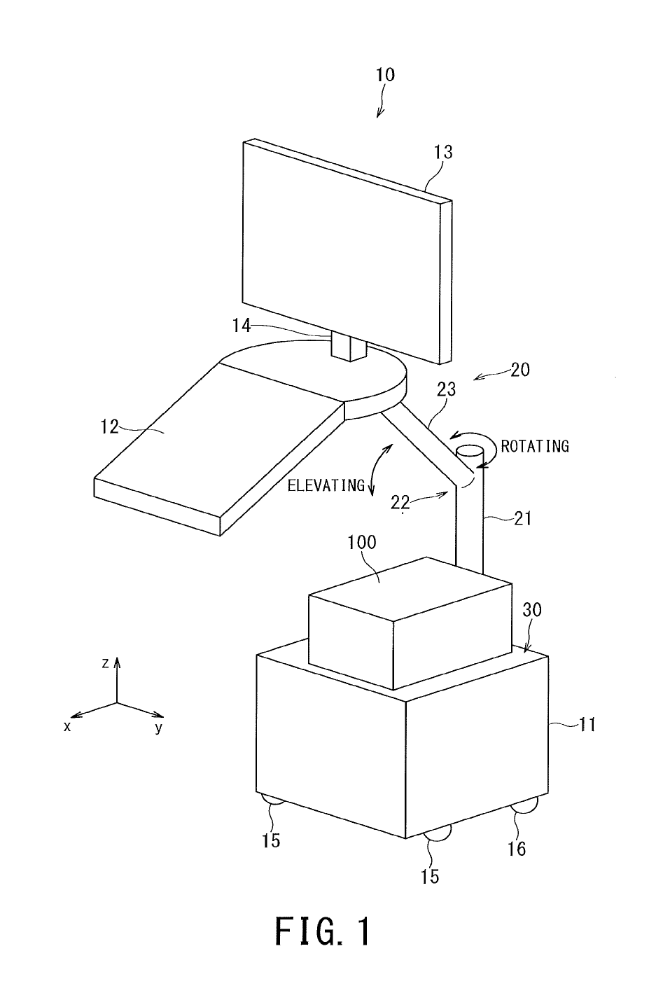

FIG. 1 is the perspective view showing the example of the ultrasonic diagnostic apparatus 10 according to the present embodiment. The ultrasonic diagnostic apparatus 10 includes the main body housing 11, the operation panel 12, the display 13, the display rotating shaft 14, the front wheel 15, the rear wheel 16, the arm supporting shaft 21, and the arm 23 supported by the arm supporting shaft 21 at the support position 22 so as to be capable of ascending and descending.

In the present embodiment, the direction perpendicular to the floor surface is defined as the z-axis, the depth direction of the main body housing 11 and the direction orthogonal to the z-axis is defined as the x-axis, and the width direction of the main body housing 11 and the direction orthogonal to the z-axis is defined as the y-axis direction (see FIG. 1).

The main body housing 11 incorporates a main body of the apparatus. The main body of the apparatus generates an ultrasonic image based on an echo signal from a subject received by an ultrasonic probe (not shown).

The operation panel 12 functions as a touch command screen and includes a display, a touch input circuit provided in the vicinity of the display, and a hard key. The touch input circuit gives the information of the indicated position on the touch input circuit by the user to the main body of the apparatus housed in the main body housing 11. The hard key includes a keyboard, a mouse, a foot switch, a track ball, various buttons, and the like. The touch input circuit and the hard key constitute an input circuit, and accept various instructions from the user of the ultrasonic diagnostic apparatus 10.

The display 13 is composed of a general display output device such as a liquid crystal display or an organic light emitting diode (OLED) display, for example, and displays ultrasound images of a living tissue image or the like generated by the main body of the apparatus incorporated in the main body housing 11. Further, the display 13 displays an image for a user of the ultrasonic diagnostic apparatus 10 to input various instructions with the use of the operation panel 12. Further, the display 13 displays notification information for the user received from the main body of the apparatus. The display 13 may be rotationally moved with respect to the operation panel 12 about the display rotation shaft 14 independently of the movement of the arm 23.

The front wheels 15 and the rear wheels 16 are wheels provided under the main body housing 11. The front wheel 15 is a freely rotatable wheel, and is composed of a pair of casters or the like, for example. The rear wheel 16 is a drive wheel connected to a drive device such as a motor, and is driven by receiving a power assist from the motor according to the operation by the user.

The arm 23 is attached to the main body housing 11 so as to be capable of ascending and descending, and so as to be pivotable about a predetermined axis. In addition, the arm 23 supports various devices such that the supported device is capable of ascending and descending, and is pivotable, according to the movement of the arm 23.

In the present embodiment, an example is shown in which the devices, that is supported by the arm 23 so as to capable of elevating (ascending and descending) and is pivotable according to the movement of the arm 23, are the operation panel 12 and the display 13 (see FIG. 1). Further, in the present embodiment, an example is shown in which the arm 23 is attached to the main body housing 11 via the arm support shaft 21.

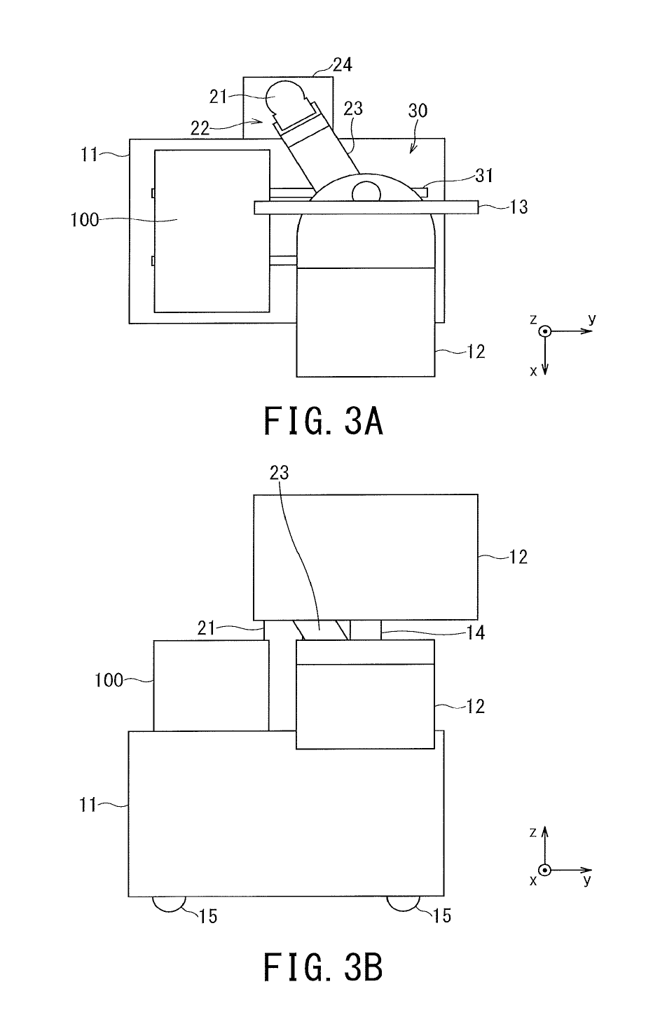

More specifically, the arm support shaft 21 is attached to the arm support base 24 (see FIG. 3A) of the main body housing 11 so as to be rotatable about the z axis. The arm 23 pivots about the z axis when the arm support shaft 21 rotates about the z axis, and the operation panel 12 and the display 13 pivot about the z axis. Further, the arm support shaft 21 supports one end of the arm 23 at the support position 22 such that the other end of the arm 23 can swing (ascend and descend) around the support position 22 in the plane including the arm support shaft 21 and the arm 23. By elevating (ascending and descending, swinging) the arm 23 around the support position 22, the operation panel 12 and the display 13 can be moved ascending and descending.

The object is placed on the surface of the table positioned in the descending direction of the arm 23. The object placed on the surface of the table includes peripheral devices 100 such as a printer and a DVD recorder, books such as instruction manuals, jelly used for ultrasonic probe, wiping papers, and the like. In the embodiment, an example is shown in which the main body housing 11 is used as the table, and the upper face 30 of the main body housing 11 is used as the surface of the table. As this type of table, besides the main body housing 11, a plate attached above the main body housing 11 can be used. Further, in the following description, an example is shown in which the object placed on the upper surface 30 of the main body housing 11 is the peripheral device 100.

As shown in FIG. 1, when the peripheral device 100 is positioned in the descending direction of the arm 23, and when the arm 23 is lowered, at least one of the arm 23 and the devices supported by the arm 23 collides with the peripheral device 100. In the following description, the devices (the operation panel 12 and the display 13) supported by the arm 23 and the arm 23 are referred to as an arm and the like 20.

### (Avoiding Mechanism)

Therefore, the ultrasonic diagnostic apparatus 10 according to the present embodiment is provided with an avoiding mechanism for changing the relative position between the peripheral device 100 and the support position 22 of the ascending and descending movement of the arm 23 in order to avoid interference between the peripheral device 100 and the arm and the like 20 due to the descending movement of the arm 23.

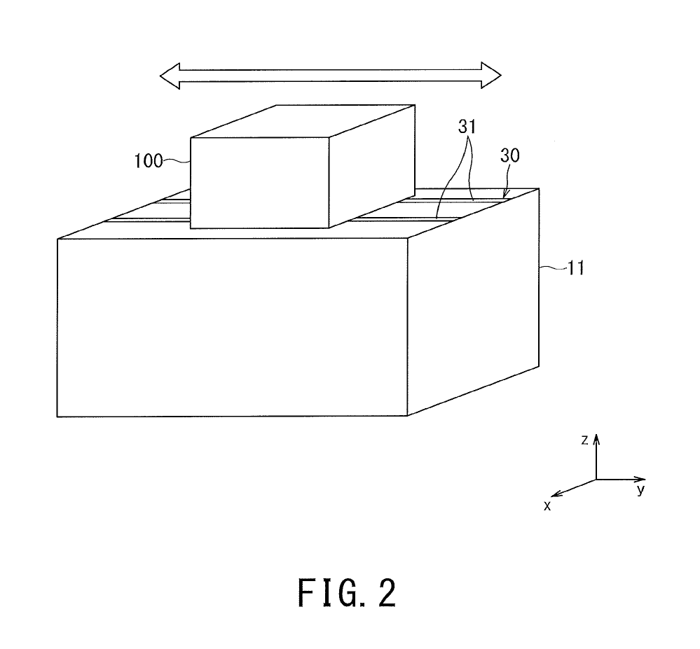

FIG. 2 is the explanatory diagram showing the configuration example of the rail 31 as an example of the avoiding mechanism.

The ultrasonic diagnostic apparatus 10 may include the rail 31 as the avoiding mechanism as shown in FIG. 2. The rail 31 is provided on the upper surface of the main body housing 11 and supports the peripheral device 100 so as to be movable along the rail 31.

FIG. 3A is the plan view showing an example of a state in which interference between the arm or the like 20 and the peripheral device 100 due to the descending movement of the arm 23 is avoided by the peripheral device 100 moving along the rail 31, and FIG. 3B is a front view of FIG. 3A.

FIGS. 2, 3A and 3B show an example in which the rail 31 is provided along the y axis on the upper surface of the main body housing 11 so as to support the peripheral device 100 being movable along the y axis. Further, the rail 31 may be provided on the upper surface of the main body housing 11 so as to movably support the peripheral device 100 along a direction inclined with respect to the y axis, and may be provided on the upper surface of the main body housing 11 along the x axis. When the rail 31 is provided along the direction inclined with respect to the y axis or along the x axis, the peripheral device 100 can move to the front, the back, or both along the rail 31.

For example, when the arm support shaft 21 is located at a position closer to one side than the center in the y axis direction of the upper surface 30, the rail 31 is preferably provided along the direction inclined with respect to the y axis so as to avoid the arm support shaft 21, or is preferably provided along the X axis on the side opposite to the arm support shaft 21 with respect to the center in the y axis direction of the upper surface 30.

Further, the rail 31 is not limited to a linear shape, and a part or all of the sections may be curved.

When the ultrasonic diagnostic apparatus 10 includes the rail 31 as the avoiding mechanism, the user moves the peripheral device 100 positioned in the descending direction of the arm 23 along the rail 31 away from the support position 22, so that interference due to the descending movement of the arm 23 between the peripheral device 100 and the arm and the like 20 (at least one of the operation panel 12, the display 13, and the arm 23) is avoided. For example, when the rail 31 is provided along the y axis as shown in FIGS. 3A and 3B, the user moves the peripheral device 100 located in the descending direction of the arm 23 along the rail 31 in the y axis direction away from the support position 22, whereby it is possible to avoid interference due to the descending movement of the arm 23 between the arm and the like 20 and the peripheral device 100.

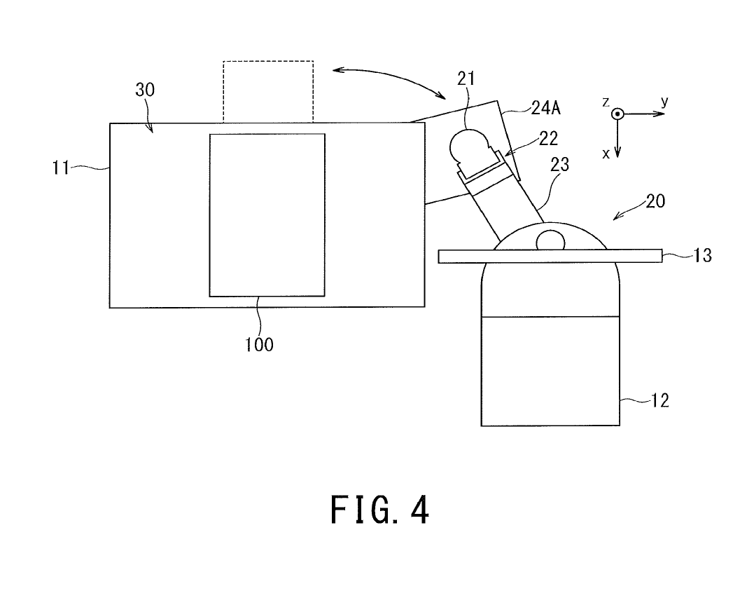

FIG. 4 is an explanatory diagram showing a configuration example of a movable arm support base 24A as another example of the avoiding mechanism.

The ultrasonic diagnostic apparatus 10 may have the movable arm support base 24A as the avoiding mechanism as shown in FIG. 4. The movable arm support base 24A supports the arm support shaft 21 so as to be rotatable around the z axis. Further, the movable arm support base 24A is mounted to the main body housing 11 so as to be movable in the xy plane, in a direction perpendicular to the z axis, by at least one of parallel movement and rotational movement with respect to the main body housing 11.

As the arm support base 24A moves in the direction perpendicular to the z axis, the arm support shaft 21 and the support position 22 move together with the arm support base 24A. Therefore, when the ultrasonic diagnostic apparatus 10 has the movable arm support base 24A as the avoiding mechanism, by moving the arm support base 24A in the direction perpendicular to the z axis, the support position 22 can be kept away from the peripheral device 100, and it is possible to avoid interference between the arm and the like 20 and the peripheral device 100 due to the descending movement of the arm 23.

When the movable arm support base 24 is used as the avoiding mechanism, the peripheral device 100 may be fixed to the upper surface 30 of the main body housing 11, or may be configured to be movable in the y-axis direction in combination with the rail 31.

It is possible to change the positional relationship between the support position 22 and the peripheral device 100 by the avoiding mechanism such that the peripheral device 100 is not positioned in the descending direction of the arm 23. Therefore, even when the peripheral device 100 is provided above the main body housing 11, it is possible to easily descending the arm 23 with avoiding interference between the arm and the like 20 and the peripheral device 100.

### (Interlocking Mechanism)

The ultrasonic diagnostic apparatus 10 may be provided with an interlocking mechanism for moving the peripheral device 100 away from the arm and the like 20 in conjunction with the pivoting of the arm 23 about the z axis.

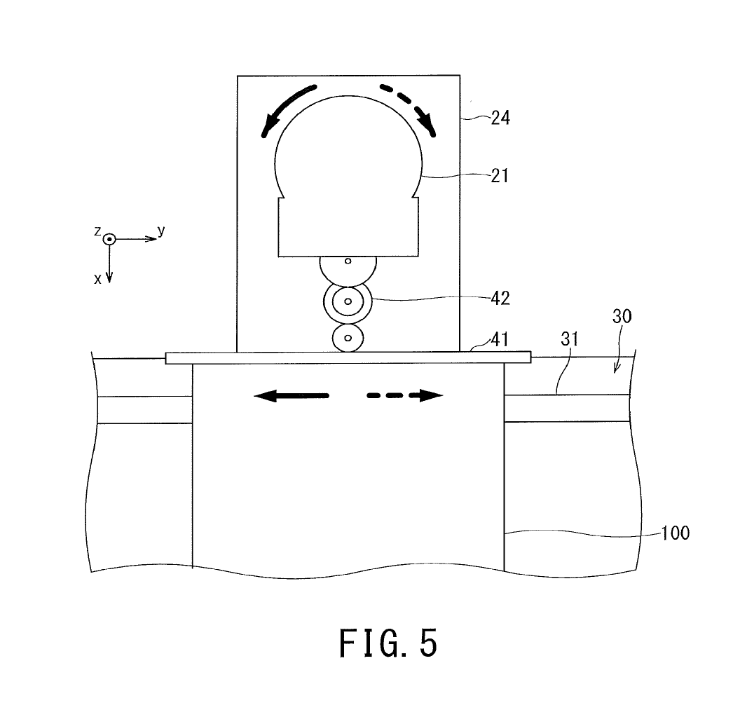

FIG. 5 is an explanatory diagram showing an example of a case where a rack-and-pinion mechanism is used as the interlocking mechanism. The arm 23 is omitted in FIG. 5.

As shown in FIG. 5, the rack and pinion mechanism may be used as the interlocking mechanism. The rack and pinion mechanism has a rack 41 and one or a plurality of gears 42.

One or the plurality of gears 42 rotate in accordance with the pivoting of the arm 23 about the z-axis. In accordance with the rotation of the gear 42, the rack 41 moves the peripheral device 100 in a direction away from the arm and the like 20 along a direction orthogonal to the z axis (in FIG. 5, along the y axis direction). For example, when the arm support shaft 21 rotates in the direction of the solid line arrow in FIG. 5, this rotation is transmitted to the rack 41 via the gears 42, and the peripheral device 100 moves in the solid line direction (the y axis negative direction). Meanwhile, when the arm support shaft 21 rotates in the direction of the broken line arrow in FIG. 5, the peripheral device 100 moves in the direction of the broken line (the y axis positive direction).

When the arm support shaft 21 rotates in the direction of the solid line arrow in FIG. 5, the arm 23 also pivots in the same direction, and the operation panel 12 pivots to the right side in FIG. 5. Therefore, according to the rack and pinion mechanism shown in FIG. 5, when the user turns the operation panel 12 to the right side in FIG. 5, the peripheral device 100 moves to the left side of the drawing by the interlocking mechanism so as to move away from the operation panel 12.

Although FIG. 5 shows an example in which the rack and pinion mechanism is used in combination with the rail 31, the rack and pinion mechanism may be used without the rail 31.

The interlocking mechanism may have a configuration to move the peripheral device 100 away from the arm and the like 20 using the position information of the arm and the like 20.

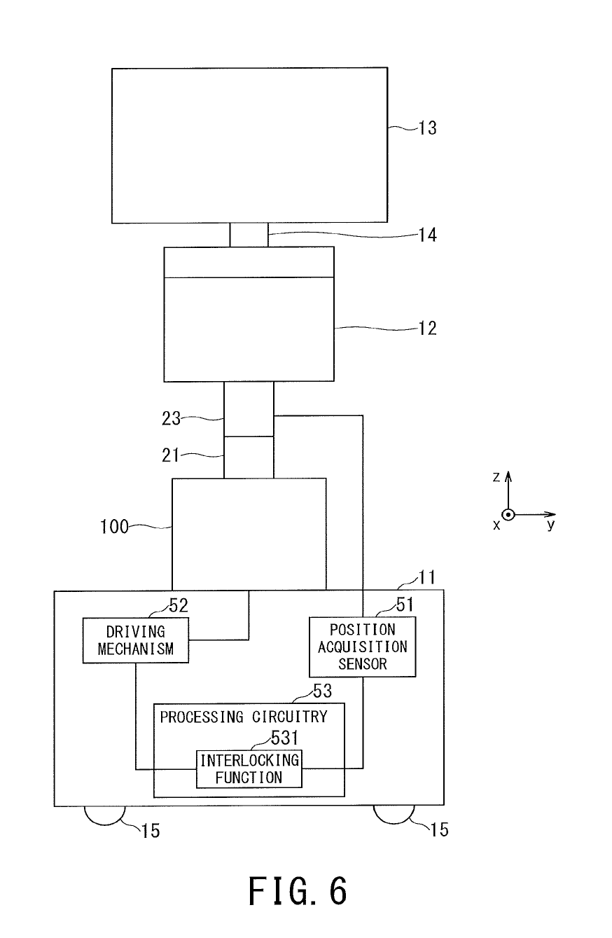

FIG. 6 is an explanatory diagram showing an example of a case where a mechanism utilizing position information of the arm or the like 20 is used as the interlocking mechanism.

As shown in FIG. 6, the interlocking mechanism using the position information of the arm and the like 20 includes, for example, a position acquisition sensor 51, a driving mechanism 52, and a processing circuitry 53.

The position acquisition sensor 51 is a sensor for acquiring position information of the arm and the like 20, and may be constituted by a position switch or the like.

The driving mechanism 52 is a mechanism for moving the peripheral device 100 and has, for example, a motor or the like. The driving mechanism 52 may use air pressure, hydraulic pressure or the like as the motive power for moving the peripheral device 100 in addition to or separately from the motor.

The processing circuitry 53 includes at least a processor and a storage medium readable by the processor. The processing circuitry 53 functions as at least an interlocking function 531 according to a program stored in the storage medium. The functions implemented by the processor are stored in the storage medium in the form of a program.

The interlocking function 531 acquires the position information of the arm and the like 20 from the position acquisition sensor 51, and controls the driving mechanism 52 so as to move the peripheral device 100 away from the arm and the like 20 in conjunction with the position information of the arm and the like 20.

According to the ultrasonic diagnostic apparatus 10 provided with the interlocking mechanism shown in FIGS. 5 and 6, the user can move the peripheral device 100 away from the arm and the like 20 by only pivoting the arm and the like 20. According to the interlocking mechanism, since the user can avoid interference between the arm and the like 20 and the peripheral device 100 without paying attention to the position of the peripheral device 100, the burden on the user can be reduced.

Further, it is preferable to provide a movement limit of the peripheral device 100 so that the peripheral device 100 does not fall from the upper surface 30 of the main body housing 11. In the case where the movement limit of the peripheral device 100 is provided and the interlocking mechanism is mounted, and when the peripheral device 100 being moved in conjunction with the pivoting of the arm 23 about the z-axis reaches the movement limit, linkage of the interlocking may be released, thereby further pivoting of the arm 23 is permitted while the peripheral device 100 places still at the movement limit.

### (Limiting Movement Mechanism)

The ultrasonic diagnostic apparatus 10 may also include a limiting movement mechanism that limits the ascending and descending movement of the arm 23 so as to reliably prevent collision between the arm and the like 20 and the peripheral device 100.

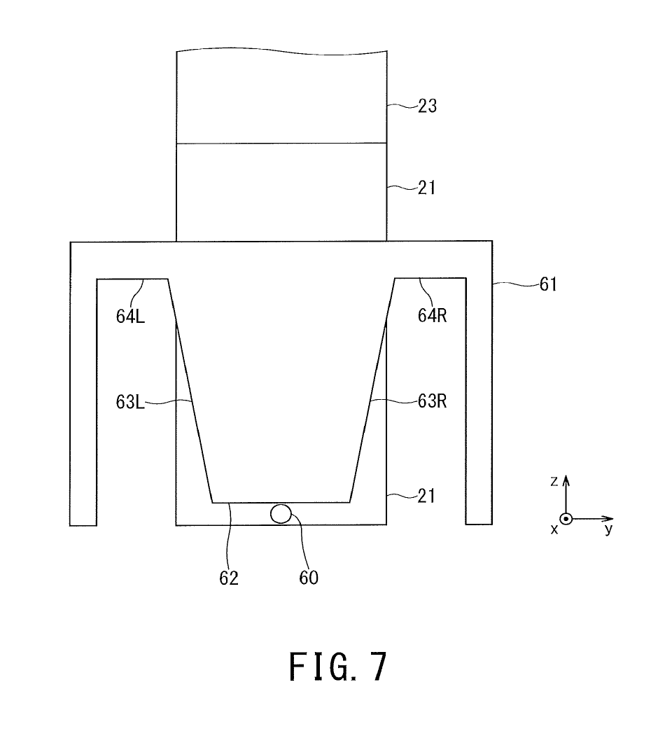

FIG. 7 is an explanatory diagram showing an example of a case where a slide pin 60 and a cam 61 are used as the limiting movement mechanism.

The slide pin 60 pivots around the z axis with the rotation of the arm support shaft 21 about the z axis.

The inner periphery of the cam 61 includes, for example, a lower side 62 located in a predetermined range in the y-axis direction including the center of the arm support shaft 21 and positioned lower in the z-axis direction, a right oblique side 63R and a left oblique side 63L extending from the lower side 62 in the upper right direction and the upper left direction, respectively, a right upper side 64R connected to the right oblique side 63R, and a left upper side 64L connected to the left oblique side 63L (see FIG. 7).

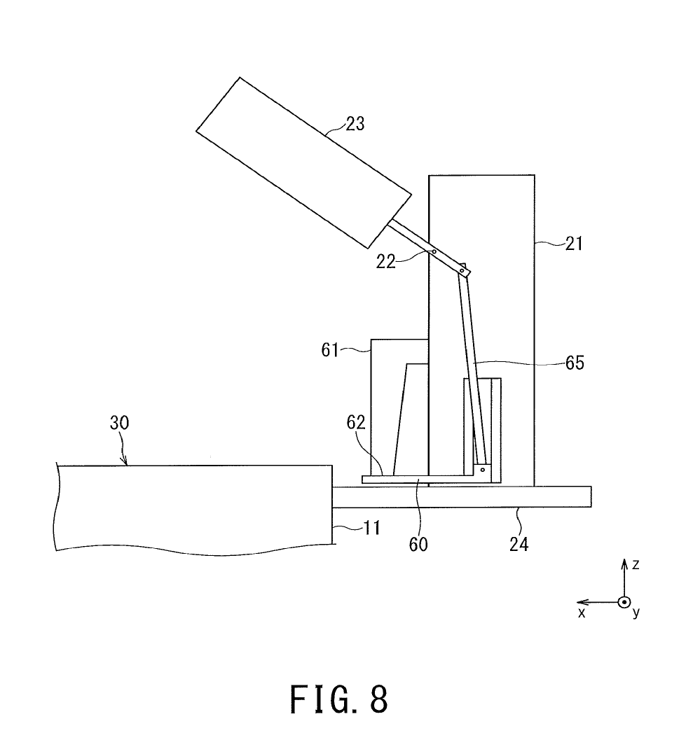

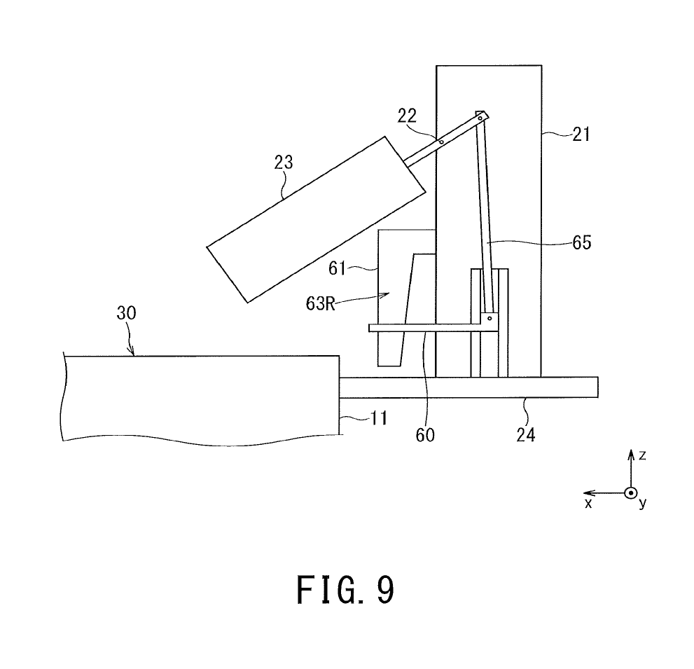

FIG. 8 is an explanatory diagram showing an example of a relationship between the arm 23 and the slide pin 60 when the slide pin 60 is within a range of the lower side 62 of the inner periphery of the cam 61. FIG. 9 is an explanatory diagram showing an example of a relationship between the arm 23 and the slide pin 60 when the slide pin 60 is within a range of the right oblique side 63R.

As shown in FIG. 8, when the slide pin 60 is within the range of the lower side 62, the slide pin 60 cannot move upward obstructed by the lower side 62. Therefore, the link 65 connected to the slide pin 60 cannot move upward either. Hence, in this case, the arm 23 cannot descend.

Meanwhile, when the slide pin 60 is outside the range of the lower side 62, the slide pin 60 is permitted to move upward. For example, as shown in FIG. 9, when the slide pin 60 is within the range of the right oblique side 63R, the slide pin 60 can move upward, with the position of the right oblique side 63 as the upper limit. Therefore, as shown in FIG. 9, as the slide pin 60 moves upward, the link 65 can move upward. Therefore, when the slide pin 60 is outside the range of the lower side 62, the arm 23 can descend about the support position 22.

That is, when the ultrasonic diagnostic apparatus 10 has the limiting movement mechanism shown in FIG. 7, the range where the descending movement of the arm 23 is prohibited can be determined according to the position, range and shape of the lower side 62, the right oblique side 63R, and the left oblique side 63L. Therefore, the ultrasonic diagnostic apparatus 10 can limit the pivoting range and the ascending and descending movement range of the arm 23 by the limiting movement mechanism, whereby interference between the arm and the like 20 and the peripheral device 100 can be easily prevented, and the burden on the user can be reduced.

### (Lock and Notification)

The ultrasonic diagnostic apparatus 10 may also include a lock mechanism that locks the wheel, or locks the peripheral device 100 and the arm and the like 20, according to the position of the arm and the like 20 and the peripheral device 100.

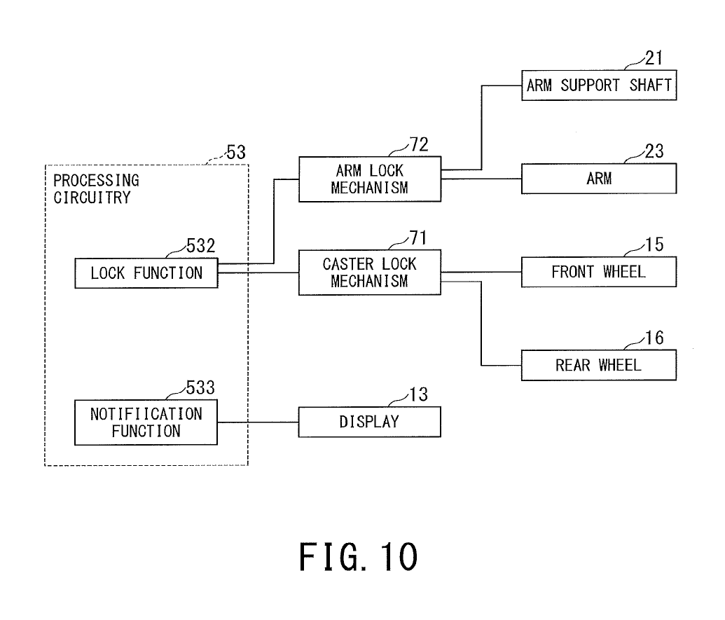

FIG. 10 is a block diagram showing a configuration example of the lock mechanism. The caster lock mechanism 71 is a mechanism for locking the front wheel 15 and the rear wheel 16 under the control of the processing circuitry 53, and is constituted by, for example, a brake or the like.

The arm lock mechanism 72 is a mechanism for inhibiting the rotation of the arm support shaft 21 and the ascending and descending movement of the arm 23 under the control of the processing circuitry 53, and is constituted by, for example, a lock pin or the like.

The processing circuitry 53 functions as a lock function 532 and a notification function 533 according to a program stored in the storage medium. It should be noted that the processing circuitry 53 may further functions as the interlocking function 531 shown in FIG. 6.

When the position of the arm 23 and the position of the peripheral device 100 are deviated from respective predetermined initial positions (see FIG. 6, for example), the lock function 532 locks the front wheel 15 and the rear wheel 16 via the wheel lock mechanism 71. Here, the predetermined initial positions are positions suitable to transfer the ultrasonic diagnostic apparatus 10.

For example, the lock function 532 unlocks the front wheel 15 and the rear wheel 16 so as to allow traveling of the ultrasonic diagnostic apparatus 10 when the arm 23 and the peripheral device 100 are in the respective predetermined initial positions. Meanwhile, when they deviate from the predetermined initial positions, the lock function 532 locks the front wheel 15 and the rear wheel 16 such that the ultrasonic diagnostic apparatus 10 cannot move, considering the risk of falling due to the fact that the center of gravity is shifted due to the peripheral device 100 being, for example, at the end of the main body housing 11, and considering the risk of collision caused by the protruding arm 23.

The notification function 533 notifies the user of the lock status of the lock function 532. For example, when the lock function 532 locks the front wheel 15 and the rear wheel 16, the notification function 533 notifies the user of the information to that effect. As a notification method, for example, a method of displaying on the touch command screen of the display 13 or the operation panel 12, a method of outputting sound via a speaker (not shown), or the like can be used. The user can understand that the ultrasonic diagnostic apparatus 10 is prohibited from traveling by notified the fact that the front wheels 15 and the rear wheels 16 are locked and can avoid unnecessary confusion by notified the fact that the front wheels 15 and the rear wheels 16 are locked.

Further, when the position of the arm 23 and the position of the peripheral device 100 are at predetermined initial positions, the lock function 532 may prohibit the rotation of the arm support shaft 21 and the ascending and descending movement of the arm 23, via the arm lock mechanism 72. In addition, when the peripheral device 100 is movable by the avoiding mechanism, the interlocking mechanism, and the like, the lock function 532 may also prohibit the movement of the peripheral device 100.

As described above, the lock function 532 unlocks the front wheel 15 and the rear wheel 16 so as to allow traveling of the ultrasonic diagnostic apparatus 10 when the arm 23 and the peripheral device 100 are in predetermined initial positions. That is, when the arm 23 and the peripheral device 100 are in the predetermined initial positions, there is a possibility that the ultrasonic diagnostic apparatus 10 travels. Therefore, in this case, the lock function 532 further prohibits the rotation of the arm support shaft 21 and the ascending and descending movement of the arm 23 such that the arm and the like 20 and the peripheral device 100 do not move during the traveling of the ultrasonic diagnostic apparatus 10. At this time, the notification function 533 may notify the user of information that the arm and the like 20 and the peripheral device 100 are locked.

Meanwhile, when the arm 23 and the peripheral device 100 are deviated from the predetermined initial positions, since the lock function 532 locks the front wheel 15 and the rear wheel 16 to prohibit the traveling of the ultrasonic diagnostic apparatus 10, the rotation of the arm support shaft 21 and the ascending and descending movement of the arm 23 can be allowed.

The lock function 532 further includes a function that, automatically returns the arm and the like 20 and the peripheral device 100 to the predetermined initial positions in accordance with the power-off operation of the ultrasonic diagnostic apparatus 10 or a predetermined button operation, and then locks the arm and the like 20 and the peripheral device 100, and unlocks of the front wheel 15 and the rear wheel 16.

According to the ultrasonic diagnostic apparatus 10 of the present embodiment, even when the peripheral device 100 is provided on the upper surface 30 of the main body housing 11, it is possible to easily prevent interference between the arm and the like 20 and the peripheral device 100 while descending the arm 23, it is possible to reduce the burden of attention when the user operates the arm 23. In addition, as compared with the case where the peripheral device 100 is provided on the side surface of the main body housing 11, the main body housing 11 can be brought closer to the bed on which the subject is placed without being disturbed by the peripheral device 100. Further, as compared with the case where the peripheral device 100 is provided on the back side of the main body housing 11, the user can easily access to the peripheral device 100, and is provided with high convenience.

According to at least one of the above-described embodiments, it is possible to easily descending the arm 23 with avoiding interference between the arm and the like 20 and the peripheral device 100 even when the peripheral device 100 is provided above the main body housing 11.

The processing circuitry 53 in the above-described embodiments is an example of the processing circuitry described in the claims. In addition, the term “processor” used in the explanation in the above-described embodiments, for instance, refer to circuitry such as dedicated or general purpose CPUs (Central Processing Units), dedicated or general-purpose GPUs (Graphics Processing Units), or ASICs (Application Specific Integrated Circuits), programmable logic devices including SPLDs (Simple Programmable Logic Devices), CPLDs (Complex Programmable Logic Devices), and FPGAs (Field Programmable Gate Arrays), and the like. The processor implements various types of functions by reading out and executing programs stored in the memory circuitry.

In addition, instead of storing programs in the memory circuitry, the programs may be directly incorporated into the circuitry of the processor. In this case, the processor implements each function by reading out and executing each program incorporated in its own circuitry. Moreover, although in the above-described embodiments an example is shown in which the processing circuitry configured of a single processor implements every function, the processing circuitry may be configured by combining plural processors independent of each other so that each processor implements each function of the processing circuitry by executing corresponding program. When a plurality of processors are provided for the processing circuitry, the memory medium for storing programs may be individually provided for each processor, or one memory circuitry may collectively store programs corresponding to all the functions of the processors.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a main body housing;
an arm mounted to the main body housing so as to be capable of ascending and descending and pivotable about a predetermined axis;
a device supported by the arm so as to be capable of ascending and descending and pivotable according to the movement of the arm;
a table having a surface which is positioned in a descending direction of the arm and the device supported by the arm, on the surface an object being placed;
an avoiding mechanism changing a relative position between the object and a support position of ascending and descending movement of the arm such that interference between the object and at least one of the arm and the device supported by the arm due to the descending movement of the arm is avoided.

2. The ultrasonic diagnostic apparatus of claim 1, wherein the object is a peripheral device associated with the ultrasonic diagnostic apparatus.

3. The ultrasonic diagnostic apparatus of claim 1, wherein
the avoiding mechanism includes a rail provided on the surface of the table and supporting the object so as to be movable in a predetermined direction, and
the object moves in the predetermined direction along the rail and moves away from the support position of the ascending and descending movement of the arm such that the interference between the object and at least one of the arm and the device supported by the arm due to the descending movement of the arm is avoided.

4. The ultrasonic diagnostic apparatus of claim 1, wherein
the avoiding mechanism supports the arm so as to be capable of ascending and descending and includes an arm support shaft attached to the main body housing so as to be rotatable about the predetermined axis and movable with respect to the main body housing in a direction perpendicular to the predetermined axis, and
the arm support shaft moves in the direction perpendicular to the predetermined axis thereby the support position of the ascending and descending movement of the arm moving away from the object such that the interference between the object and at least one of the arm and the device supported by the arm due to the descending movement of the arm is avoided.

5. The ultrasonic diagnostic apparatus of claim 1, further comprising an interlocking mechanism for moving the object away from the arm and the device supported by the arm in conjunction with the position of the arm and the device supported by the arm.

6. The ultrasonic diagnostic apparatus of claim 5, wherein the interlocking mechanism includes a gear that rotates in response to the pivoting movement of the arm about the predetermined axis and a rack for moving the object in a direction away from the arm and the device supported by the arm in accordance with the rotation of the gear.

7. The ultrasonic diagnostic apparatus of claim 5, wherein the interlocking mechanism includes a driving mechanism for moving the object, a sensor configured to acquires position information of at least one of the arm and the device supported by the arm, and a processing circuitry configured to control the driving mechanism so as to move the object away from the arm and the device supported by the arm in conjunction with the position information.

8. The ultrasonic diagnostic apparatus of claim 1, further comprising a limiting movement mechanism limiting a range of the ascending and descending movement of the arm according to the position of the object.

9. The ultrasonic diagnostic apparatus of claim 1, further comprising:
a wheel provided on the main body housing; and
a processing circuitry configured to lock the wheel when a position of the arm and a position of the object deviate from respective predetermined initial positions.

10. The ultrasonic diagnostic apparatus of claim 9, wherein the processing circuitry locks movement of the arm so as to prohibit the ascending and descending movement and a pivoting movement when the position of the arm and the position of the object are at the respective predetermined initial positions.

11. The ultrasonic diagnostic apparatus of claim 9, wherein the processing circuitry notifies a user of lock status.

12. The ultrasonic diagnostic apparatus of claim 1, wherein the device supported by the arm includes at least one of a display and an operation panel.

13. The ultrasonic diagnostic apparatus of claim 1, wherein the table is the main body housing, and the surface is the upper surface of the main body housing.
